(19) United States
(12) Patent Application Publication
(10) Pub. No.: US 2003/0216793 A1
(43) Pub. Date: Nov. 20, 2003

(54) Implantable antenna for use with an implantable medical device

(75) Inventors: Anders Karlsson, Dalby (SE); Hans Abrahamson, Stockholm (SE); Magnus Lindberg, Sundbyberg (SE)

Correspondence Address: SCHIFF HARDIN & WAITE, 6600 SEARS TOWER, 233 S WACKER DR, CHICAGO, IL, 60606-6473, US

(73) Assignee: St. Jude Medical AB

(21) Appl. No.: 10/364,479

(22) Filed: Feb. 11, 2003

(30) Foreign Application Priority Data
May 17, 2002 (SE) 0201490-0

(51) Int. Cl. A61N 1/32

(52) U.S. Cl. 607/60

(57) ABSTRACT

For wireless data exchange between an implantable medical device and an extracorporal unit by means of an antenna arrangement for communicating radio frequent signals, the antenna arrangement includes a patch plane having a perimeter, a ground plane of an electrically conducting material and a dielectric substrate filling a volume between the patch plane and the ground plane. The ground plane has an area which is larger than the patch plane and is located in relation to the patch plane such that a perpendicular projection of the patch plane onto the ground plane falls entirely within the ground plane. A grounding member connects the patch plane electrically to the ground plane at a first segment of the perimeter. The substrate has a comparatively high relative permittivity and extends a well-defined distance outside the volume between the patch plane and the ground plane with respect to at least one second segment of the perimeter. A non-conducting region thus is created outside the patch plane and therefore only a relatively small amount of electromagnetic losses will occur in this region.

### BACKGROUND OF THE INVENTION

1. Field of the Invention

The present invention relates generally to an implantable medical device of the type associated with means for wireless data exchange with an extracorporal unit. More particularly the invention relates to an antenna arrangement for such data exchange.

2. Description of the Prior Art

It is desirable for patient comfort for an implantable medical device (IMD) to remain in the body for as long a time as possible without explantation. Therefore, it is advantageous for the IMD to communicate with an external unit while the device is still implanted. Conventionally, status information and measurement parameters have been read out from implanted devices via an inductive telemetry interface. This type of interface is also usable for transferring of data in the opposite direction, for example when adjusting parameters pertaining to the operation of the IMD or when updating the device's program code to a newer version. However, the inductive interface requires a relatively short distance (on the order of centimeters) between the implanted device and the extracorporal unit with which it communicates. This, in turn, may be inconvenient for the patient as well as impractical for the personnel conducting the procedure. Moreover, the maximum data rate for an inductive interface is relatively low, which results in practical limitations as to the amount of data that can be communicated.

Therefore efforts are now being made in order to find alternative solutions to the inductive telemetry interface for communicating with an IMD. For instance, the American Federal Communications Commission (FCC) and the European Telecommunications Standard Institute (ETSI) have proposed a dedicated radio frequency range in the band 402-405 MHz for communication with IMDs. Additionally, the patent literature includes examples of solutions for accomplishing a radio link between an implanted device and an external unit.

U.S. Pat. No. 6,115,636 describes a telemetry system for implantable devices with the human body functioning as a radio antenna for the implanted device. The housing of the device is used to accomplish a coupling between the patient's body and the device and thus makes possible the transmission of a modulated electromagnetic signal.

U.S. Pat. No. 5,626,630 discloses a telemetry solution involving a quasi-passive implanted transponder and a repeater station to be worn externally by the patient. The repeater station operates as a relay between an IMD and a remote monitoring station to provide a communications link with a high data rate. The transponder includes a couple of microstrip radio antennae, which are resonant at one half of the signal wavelength used.

U.S. Pat. No. 5,861,019 shows another example of an IMD equipped with a microstrip radio frequency telemetry antenna. The antenna is formed on or within the exterior surface of the device housing. The antenna extends over the entire available surface area of the housing and is covered by a radome layer to ensure that the patch is electrically insulated from body fluids and tissue.

Although the above-mentioned solutions generally represent improvements in comparison to an inductive link regarding data rate and communication range, they fail to present a telemetry solution with satisfactory power efficiency and sufficiently small physical dimensions of the antenna. The size of the antenna is a major concern because the antenna is to be implanted along with the IMD into a human body. For the best patient comfort, a smallest possible physical size of the antenna is desired. However, low power consumption is at least as important. A change of batteries requires explantation of the device, and therefore should be avoided as long as possible.

The article C. Furse, “Design of an Antenna for Pacemaker Communication,” Microwaves and RF, March 2000, pp 73-76 describes a microstrip antenna for radio signals at a frequency of 433 MHz. It is proposed that an L-, U- or spiral-shaped patch antenna be arranged on top of a 6 mm thick Teflon® substrate. The antenna's physical dimensions thus are limited, however its geometry becomes intricate. Moreover, the design is inclined to cause large ohmic losses and consequently not be particularly power efficient.

### SUMMARY OF THE INVENTION

It is an object of the present invention to provide an improved implantable antenna solution for an IMD which avoids or alleviates the aforementioned problems.

This object is achieved in an antenna arrangement in accordance with the invention, for communicating radio frequency signals with an implantable medical device, having a patch plane adapted for placement on the medical device which is capable of exchanging modulated electromagnetic energy with a surrounding transmission medium, a ground plane composed of an electrically conducting material and having an area which is larger than the patch plane, the ground plane being located relative to the patch plane so that a perpendicular projection of the patch plane onto the ground plane falls entirely within the ground plane, a dielectric substrate filling a volume between the patch plane and the ground plane, a grounding member electrically connecting the patch plane to the ground plane, with the grounding member being attached to a first segment of the perimeter of the patch plane, and wherein the substrate has a comparatively high relative permittivity and that the substrate extends a well-defined distance outside the volume between the patch plane and the ground plane with respect to at least one second segment of the perimeter.

An important advantage attained by such extension of the substrate is that a non-conducting region is thereby obtained outside the patch plane. This, in turn, guarantees that there will be only relatively small electromagnetic losses in this region. The electric fields closest to the patch are quasi-static and hence very large. In a conducting medium (such as the human body), however, the dissipated power density is proportional to the square of the electric field. This means that if the region closest to the patch plane were occupied with a conducting material a large amount of the power leaving the patch would be used for heating the region closest to the patch. From a power efficiency point of view, this is an undesired effect, which is avoided by the inventive antenna arrangement. A high relative permittivity is desirable because the required length of the patch plane is inversely proportional to the square root of the relative permittivity. Consequently, a high relative permittivity makes it possible to achieve an antenna with small physical dimensions. For example, a substrate of a ceramic material is advantageous, since ceramic materials are available with relative permittivities up to around 100. Alternatively, the substrate may contain a laminated thick-film structure including layers of aluminum oxide. Such substrate can be designed with a relative permittivity of approximately 1000.

In a preferred embodiment of the inventive antenna arrangement, the substrate overlaps a section of the patch plane along (at least one) sub-segment of the at least one second segment. An advantage thus attained is that the losses in the dissipative medium represented by the body tissue are further reduced.

In another preferred embodiment of the antenna arrangement of the invention, the grounding member includes a conducting plane, which forms a junction with the patch plane along the first segment. A conducting plane connected along the entire length of the first segment results in a uniform distribution of the electric and the magnetic fields over the antenna section. If however, the conducting plane is less extended than the patch plane the resonance frequency for the antenna decreases to a corresponding extent, which may be desirable in some applications.

In another preferred embodiment of the inventive antenna arrangement, the grounding member is substantially perpendicular to the ground plane. It is not necessary for the antenna function for the grounding member to be entirely perpendicular to the ground plane, however such orientation results in a more uniform distribution of the electric and the magnetic fields over the antenna section. Feeding the antenna and extracting a received signal from the antenna are thereby also facilitated. For instance, the antenna arrangement may include a signal member adapted to transmit a radio frequency signal between the patch plane and a radio transceiver. The signal member extends through the ground plane via an electrically insulated feed-through at a particular distance from the grounding member. The signal member includes an element that is substantially perpendicular to the ground plane and is either electrically insulated from the patch plane or connected to a side of the patch plane, which faces toward the ground plane.

The above object also is achieved in accordance with the invention in an implantable medical device as described initially having an inventive antenna arrangement.

In a preferred embodiment of the implantable medical device of the invention, the antenna arrangement is located in a recess of an outer side of a casing to the device. This is advantageous because a slim device design is thus made possible.

According to a first alternative preferred embodiment of the inventive implantable medical device, the antenna arrangement is molded into a non-conductive encapsulation, such that the ground plane is electrically insulated from the device's casing. This may be desirable in applications where an absolute control of the ground plane properties is required.

In a second alternative preferred embodiment of the inventive implantable medical device, the device's casing instead constitutes the ground plane. Again, this results in a slim and uncomplicated design of the device.

In another preferred embodiment of the implantable medical device of the invention, the patch plane is tilted with respect to the ground plane, such that the arrangement attains horn antenna properties. This, in turn, results in an antenna with an increased bandwidth, since it facilitates the propagation of the outgoing electromagnetic waves. Moreover, the efficiency of the antenna is enhanced due to an increased radiation resistance. Preferably, the patch plane is tilted such that the distance between the patch plane and the ground plane is shortest at a point where the grounding member connects to the patch plane.

In another preferred embodiment of the implantable medical device of the invention, the casing constitutes the ground plane and the ground plane includes a chamfer. This chamfer is oriented such that the distance between the patch plane and the ground plane extends gradually along a slope of the chamfer and the distance between the ground plane and the patch plane is largest at an edge side farthest away from the grounding member. Again, this results in an antenna arrangement having properties similar to that of a horn antenna.

A general advantage with the present invention is that simple antenna geometry is provided, which can be manufactured by means of a fairly uncomplicated production process. Furthermore, the inventive antenna geometry results in relatively low ohmic losses.

Although the inventive solution is primarily intended for cardiac devices, such as pacemakers and defibrillators, the invention is equally applicable to any type of implantable medical device, for example drug pumps, neurostimulators, gastric stimulators, muscle stimulators and hemodynamic monitors.

### BRIEF DESCRIPTION OF THE DRAWINGS

FIG. 1 schematically shows an IMD which communicates with an extracorporal unit over a wireless interface according to the invention.

FIG. 2 illustrates the general design of the proposed antenna arrangement.

FIGS. 3a-h show top views of alternative patch plane shapes according preferred embodiments of the invention.

FIG. 4 shows a side view of an antenna arrangement according to a first preferred embodiment of the invention.

FIG. 5 shows a side view of an antenna arrangement according to a second preferred embodiment of the invention.

FIG. 6 shows a side view of an IMD including an antenna arrangement according to a first preferred embodiment of the invention,

FIG. 7 shows a side view of an IMD including an antenna arrangement according to a second preferred embodiment of the invention.

FIG. 8 shows a side view of an IMD including an antenna arrangement according to a third preferred embodiment of the invention.

FIG. 9 illustrates an IMD including an antenna arrangement with a tilted patch plane according to a first preferred embodiment of the invention.

FIG. 10 illustrates an IMD including an antenna arrangement with a chamfered ground plane according to a second preferred embodiment of the invention.

FIG. 11 illustrates an IMD including an antenna arrangement with a convex patch plane and a curved ground plane according to a third preferred embodiment of the invention.

### DESCRIPTION OF PREFERRED EMBODIMENTS

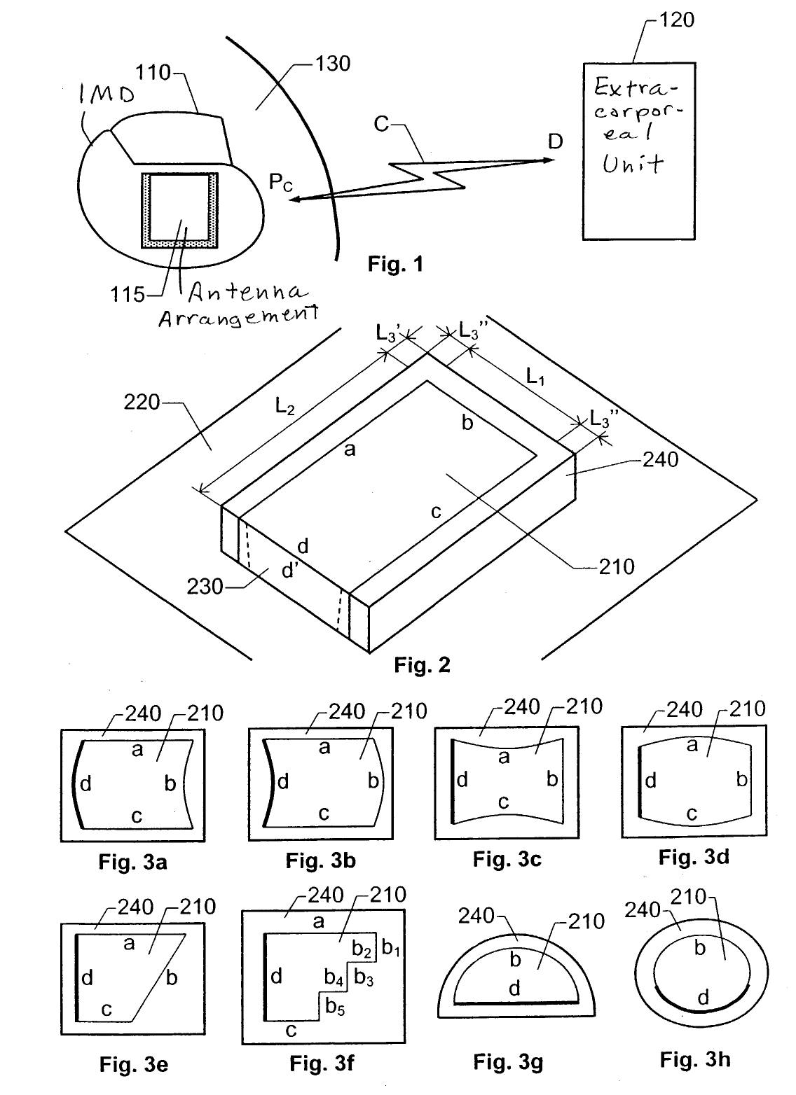

A wireless communication scenario involving an IMD 110 and an extracorporal unit 120 is shown schematically in FIG. 1. Data D and Pc may here be transferred in both directions between the IMD 110 and the extracorporal unit 120 over a radio link C. Typically, parameter settings or updating software Pc is sent from the unit 120 to the IMD 110 while measurement data D is sent in the opposite direction for various monitoring and diagnostic purposes.

According to the invention, an antenna arrangement 115 in the IMD 110 is used to exchange modulated electromagnetic energy with the surrounding transmission medium, i.e. the relevant body tissue 130. Specifically, this means that outgoing radio signals D are coupled through the body 130 and out into the contiguous environment, such as the air. The radio signals D then continue to propagate via the air to the extracorporal unit 120 where they are received, for example by a conventional radio antenna. Correspondingly, incoming radio signals Pc that are received by the IMD 110 have initially been emitted by the unit 120, propagated through the air and penetrated through the body tissue 130 to reach the antenna arrangement 115.

However, transmitting the same radio signals through a lossy medium, such as the human body, and through a much less lossy medium, such as the air, is a far from trivial task. The transmission media's different permittivities must, of course, be considered. The body tissue's properties may be approximated as being close to those of water in respect to permittivity. This means that the wavelength inside the body is roughly 1/9th to ⅛th of the wavelength in free air. For example, if the radio frequency is 403 MHz, the wavelength becomes 0.74 m in free air and around 9.2 cm inside the body.

A patch type of antenna is included in the antenna arrangement 115 according to the invention. Such antennae are advantageous because they are easy to build using printed circuit technology. Furthermore, the design results in a comparatively low weight antenna, which has small overall dimensions and is relatively inexpensive to manufacture. Preferably, the antenna design is a planar inverted F-antenna (PIFA), which is resonant at one quarter of a wavelength. I.e. the above example would require a PIFA that is 2.3 cm long. In order to obtain a power efficient antenna, the substrate should have a permittivity that results in a wavelength in the substrate which is approximately equal to the wavelength in the surrounding tissue, i.e. the human body.

The general design of the inventive antenna arrangement 115 is illustrated in FIG. 2. A patch plane 210 is here adapted to exchange modulated electromagnetic energy with a surrounding transmission medium, i.e. normally body tissue. The depicted patch plane 210 has a general rectangular shape with dimensions L₁ and L₂ and is thus circumscribed by a perimeter including four separate edges a, b, c and d respectively. However theoretically, any alternative shape of the patch plane 210 is conceivable provided that its physical dimensions are adapted to the specific application. It is the geometry of the patch plane 210 that determines the antenna's impedance and bandwidth. Various examples of alternative patch plane shapes will be discussed below with reference to FIGS. 3a-h.

Returning to FIG. 2, a ground plane 220 of an electrically conducting material is located below the patch plane 210. The ground plane 220 has a larger area than the patch plane 210 and is positioned in relation to the patch plane 210, such that a perpendicular projection of the patch plane 210 onto the ground plane 220 falls entirely within the ground plane 220. A dielectric substrate 240 with a comparatively high relative permittivity, say εᵣ=70 or more, fills the entire volume between the patch plane 210 and the ground plane 220. The substrate 240 also extends a well-defined distance L₃′ and/or L₃″ outside the volume between the patch plane 210 and the ground plane 220 with respect to at least one the patch plane's 210 edges a, b and c, preferably at least the edge d.

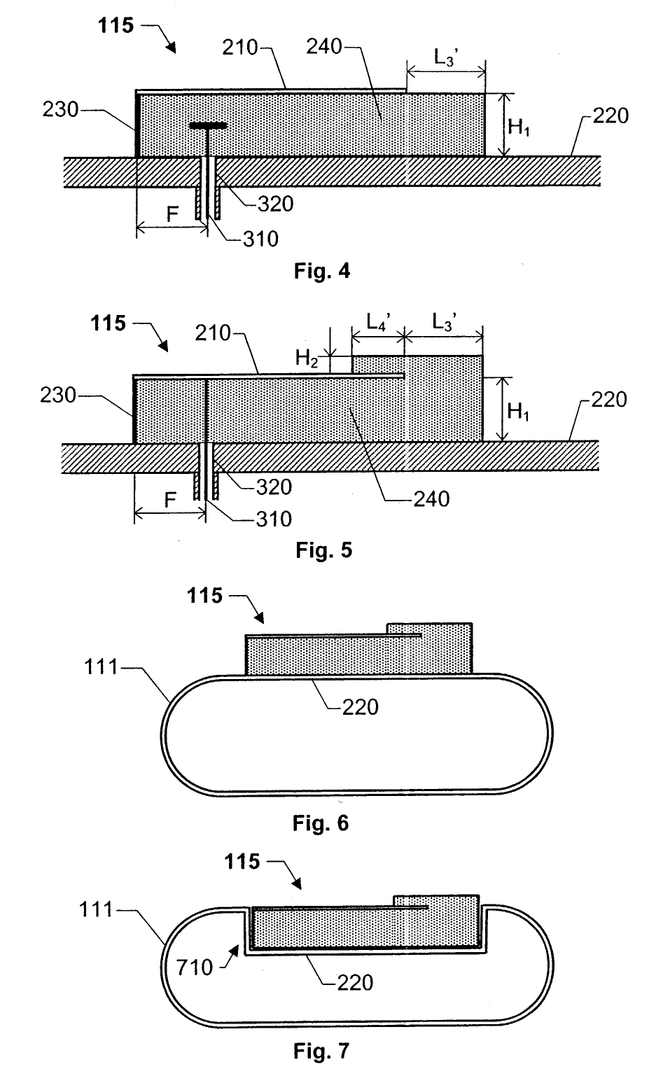

It is important that the substrate 240 extends L₃′, L₃″ outside the patch plane 210 because thereby a non-conducting region is obtained outside the patch plane 210, where otherwise electrically conducting body tissue would be located. The extension of the substrate 240 guarantees that there will only be a relatively small amount of electromagnetic losses in these regions. The electric fields closest to the patch plane 210 are quasi-static and hence comparatively large. In a conducting medium the dissipated power density is proportional to the square of the electric field. This means that had the region closest to the patch plane 210 been occupied with a conducting material, such as body tissue, a large amount of the power leaving the antenna would be used for heating the region closest to the patch plane 210. However, the distance L₃′, L₃″ between the patch plane 210 and the closest conducting region established by the extended substrate 240 accomplishes a volume over which the electric field may decrease without losses. The proposed design is thus very advantageous with respect to power efficiency. Further details pertaining to the relevant dimensions will be discussed below with reference to FIGS. 4 and 5.

According to the above-described embodiment of the invention, a grounding member 230 connects the patch plane 210 electrically to the ground plane 220. Preferably, the grounding member 230 includes a conducting plane, which forms a junction d′ with the patch plane 210 along a substantial part of the edge d of the patch plane. It is generally most preferred if the conducting plane of the grounding member 230 extends along the entire length of the edge d, i.e. a distinct segment of the perimeter of the patch plane 210. A conducting plane whose width equals the edge d of the patch plane 210 results in a uniform distribution of the electric and the magnetic fields over the antenna section. However, the conducting plane of the grounding member 230 needs only extend along a sub-segment of the edge d. If however, the conducting plane is less extended than the patch plane the resonance frequency for the antenna decreases to a corresponding extent, which may be desirable in some applications. FIG. 2 shows a grounding member 230 with a shortened conducting plane by means of dashed lines. Note that the conducting plane edges may have arbitrary orientation in relation to the ground plane 220.

As already mentioned, the dielectric substrate 240 preferably has a high relative permittivity. The required length L₂ of the patch plane 210 is namely inversely proportional to the square root of the substrate's 240 relative permittivity. Consequently, a high relative permittivity reduces the patch plane's 210 physical dimensions L₁ and L₂. A ceramic material may be used to accomplish a substrate 240 with a moderate relative permittivity, in the order of 100. If higher values of the relative permittivity are required, the substrate 240 should preferably include a laminated thick-film structure, for instance with layers of aluminum oxide. Thereby, relative permittivities up to 1000 may be accomplished.

FIGS. 3a-h illustrate examples of alternative patch plane 210 shapes according embodiments of the invention. The first four FIGS. 3a-d represent patch plane 210 shapes that result in comparatively narrow-band antennae. Namely, the perpendicular distance between the perimeter segment d being attached to the grounding member and an opposite non-grounded perimeter segment is here constant. FIGS. 3e-h, however, represent patch plane 210 shapes that result in antennae, which are operable over a broader bandwidth because here the perpendicular distance between the perimeter segment d being attached to the grounding member and an opposite non-grounded perimeter segment varies. For example, the patch plane 210 shape shown in FIG. 3f provides an antenna adapted to operate over three distinct frequency bands, which are given by the perpendicular distances between the perimeter segment d and the perimeter segments b₁, b₃ and b₅ respectively, whereas the patch plane 210 shape shown in FIG. 3e provides an antenna adapted to operate over one comparatively wide frequency band.

In a preferred embodiment of the invention, the grounding member 230 is substantially perpendicular to the ground plane 220. This namely results in a uniform distribution of the electric and the magnetic fields over the antenna section. Moreover, feeding the antenna and extracting a received signal from the antenna is facilitated by such design. This matter will be discussed in further detail below with reference to FIGS. 4 and 5.

FIG. 4 shows a side view of an antenna arrangement 115 according to a first preferred embodiment of the invention. The arrangement 115 includes a signal member 310, which is adapted to transport a radio frequency signal between the patch plane 210 and a radio transceiver. I.e. the signal member 310 may be used either to feed a radio signal to the patch plane 210 or to extract a received radio signal therefrom. The signal member 310 has a cylindrical element, which is substantially perpendicular to the ground plane 220 and extends through the ground plane 220 via an electrically insulated feedthrough 320. The feedthrough 320 is positioned at a distance F from the grounding member 230, where F is selected with respect to the substrate 240 and the properties of the radio signal used, such that a desired impedance of the antenna is obtained. In this embodiment, the signal member 310 ends with a radiating element, which is electrically insulated from the patch plane 210.

The substrate 240 has a thickness H₁ and extends a distance L₃′ outside the edge b of the patch plane 210. According to a preferred embodiment of the invention, the distance L₃′ is proportional to the length L₂ of the patch plane 210, such that L₃′ lies in the interval 10-30% of L₂, and preferably around 20% of L₂. According to a preferred alternative of this embodiment of the invention, the substrate 240 also extends a distance L₃″ outside the edges a and c of the patch plane 210 (see FIG. 2). The distance L₃″ is likewise proportional to the length L₂, such that L₃″ lies in the interval 10-20% of L₂, preferably at least 15%.

The specific relationships L₃′-to-L₂ and L₃″-to-L₂ can be explained as follows. The efficiency of a transmitting antenna operating in air is defined by the ratio between the power delivered to the antenna and the power radiated from the antenna. The sum of the losses in the substrate and in the conducting parts determines the antenna's efficiency. For an implanted transmitting antenna, however, it is relevant to define the overall efficiency of the antenna system as the ratio between the power delivered to the antenna and the power radiated from the body. Thus, the efficiency is now determined both by the losses in the antenna and in the lossy medium represented by the body tissue. The ohmic power loss density Pₗₒₛₛ (W/m³) in a conductive medium is given by the expression:

$${P}_{loss} = \frac{1}{2} J \cdot {E}^{*} = \frac{1}{2} σ {\vert E \vert}^{2}$$

where σ is the conductivity of the medium and E is the electric field. A tissue consisting of muscles has a relatively high conductivity (σ≈1,4 s/m at a frequency around 400 MHz including dielectric losses). Hence, the efficiency of the antenna system becomes rather low if the antenna is surrounded by muscle tissue. As is apparent from the above expression, the power loss density Pₗₒₛₛ is high in regions where the electric field is high. The electric field shows high values in the near zone of the patch plane, since in that region the field is dominated by the non-radiating near field. In the PIFA-case, the near zone is the region close to the outside of the non-grounded segments of the patch plane perimeter (i.e. for example the edges a, b and c in the FIG. 2).

FIG. 5 shows a side view of an antenna arrangement 115 according to a second preferred embodiment of the invention. In this embodiment, the signal member 310, is electrically connected to the side of the patch plane 210, which faces towards the ground plane 220. The signal member 310 is also here substantially perpendicular to the ground plane 220 and extends through the ground plane 220 via an electrically insulated feedthrough 320. The feedthrough 320 is positioned at a distance F from the grounding member 230, where F is selected with respect to the substrate 240 and the properties of the radio signal used, such that a desired impedance of the antenna is obtained.

The substrate 240 has a thickness H₁ and does here not only extend a distance L₃′ outside the edge b of the patch plane 210, however it also overlaps L₄′ a section of the patch plane 210 along at least one edge, for example the edge b. In analogy with the embodiment of the invention described above with reference to FIGS. 2 and 3, the distance L₃′ is preferably proportional to the length L₂ of the patch plane 210. The same is basically true for the overlap L₄′, however it is sufficient if L₄′ corresponds to 10% of L₂. The thickness H₂ of the overlap should correspond to at least 5% of the length L₂ of the patch plane 210. In any case, the overlap assists in further reducing the losses in the dissipative medium represented by the body tissue.

The features of the signal member 310 being electrically connected to the patch plane 210 (as shown in FIG. 5) respective being insulated from the patch plane 210 (as shown in FIG. 4) and the substrate 240 overlapping L₄′, H₂ a section of the patch plane 210 (as shown in the FIG. 4) respective merely extending a well-defined distance L₃′ (as shown in the FIG. 4) may be combined arbitrarily. The specific combinations of these features as shown in FIGS. 4 and 5 have merely been chosen for illustrating purposes.

FIG. 6 shows a side view of an IMD including an antenna arrangement 115 according to a first preferred embodiment of the invention. The arrangement 115 is located on the outer surface of an IMD casing, such that the casing 111 constitutes the ground plane 220. A straightforward design being relatively inexpensive to manufacture is thus made possible.

FIG. 7 shows a side view of an IMD including an antenna arrangement 115 according to a second preferred embodiment of the invention. Also here the casing 111 to the device constitutes the ground plane 220. However, the arrangement 115 is now located in a recess 710 of an outer side of the casing 111. This type of design is often preferable, since due to the fact the electric field decreases with increasing substrate thickness, a relatively thick antenna is also comparatively power efficient. By mounting antenna arrangement 115 in the recess 710 it is thus possible to have a rather thick antenna with a high efficiency and at the same time accomplish a slim device.

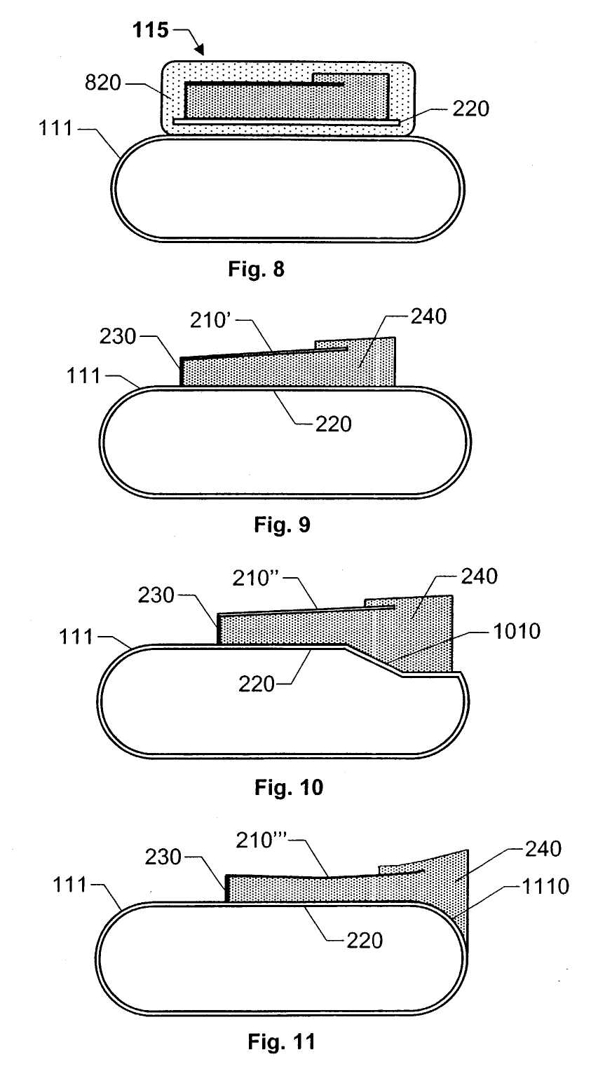

FIG. 8 shows a side view of an IMD including an antenna arrangement 115 according to a third preferred embodiment of the invention, where the entire arrangement 115 is molded into a non-conductive encapsulation 820. The encapsulation 820 insulates a ground plane 220 in the arrangement 115 electrically from the casing 111 to the device. Thereby a complete control of the ground plane's 810 properties can be obtained.

FIG. 9 illustrates an IMD including an antenna arrangement 115 with a tilted patch plane 210′ according to a first preferred embodiment of the invention. Again, the casing 111 to the device constitutes the ground plane 220. The patch plane 210′, however, is tilted with respect to the ground plane 220, such that the patch plane 210′ is parallel to the ground plane 220 exclusively in one dimension. Hence, the arrangement 115 attains signal properties that resemble those of a horn antenna. For instance, the bandwidth increases. The radiation resistance also raises, which results in a decreased electric field and an enhanced power efficiency.

Preferably, the patch plane 210′ is tilted such that the distance between the patch plane 210′ and the ground plane 220 is shortest at a point where a grounding member 230 connects to the patch plane 210′.

FIG. 10 illustrates an IMD including an antenna arrangement 115 with a chamfered ground plane 220 according to a second preferred embodiment of the invention. Again, the casing 111 to the device constitutes the ground plane 220. Although not actually necessary in this embodiment the patch plane 210″ is also tilted with respect to the ground plane 220, such that the patch plane 210″ is parallel to the ground plane 220 exclusively in one dimension. More important however, a portion of the casing 111, which constitutes the ground plane 220 here includes a chamfer 1010. This chamfer 1010 is oriented such that the distance between the patch plane 210″ and the ground plane 220 extends gradually along a slope of the chamfer 1010. Furthermore, the chamfer 1010 is placed such that the distance between the ground plane 220 and the patch plane 210″ is largest at the patch plane edge (edge b in FIG. 2) being furthest away from a grounding member 230. This design accentuates the horn antenna properties and the thus attained advantages mentioned above.

FIG. 11 illustrates an IMD including an antenna arrangement 115 with a convex patch plane 210′″ and a curved ground plane 220 according to a third preferred embodiment of the invention. Here, the patch plane 210′″ has a general curved profile with a convex face towards the ground plane 220. Again, the casing 111 to the device constitutes the ground plane 220. In this embodiment, the substrate 240 extends over the curved side 1110 of the casing 111, such that the substrate 240 forms a tapered section towards the curved side 1110, which is furthest away from a grounding member 230. Thereby, an alternative horn antenna design is accomplished.

Although modifications and changes may be suggested by those skilled in the art, it is the intention of the inventors to embody within the patent warranted hereon all changes and modifications as reasonably and properly come within the scope of their contribution to the art.

We claim as our invention:

1. An antenna arrangement for communicating radio frequency signals with an implantable medical device, comprising:
a patch plane adapted for placement on an implantable medical device and adapted to exchange modulated electromagnetic energy with a surrounding transmission medium, the patch plane having a perimeter formed by a plurality of segments;
a ground plane of an electrically conducting material having an area which is larger than the patch plane, the ground plane being located in relation to the patch plane such that a perpendicular projection of the patch plane onto the ground plane falls entirely within the ground plane;
a dielectric substrate filling a volume between the patch plane and the ground plane;
a grounding member electrically connecting the patch plane to the ground plane, the grounding member being attached to a first of said segments of the perimeter; and
the substrate having high relative permittivity and extending a distance outside the volume between the patch plane and the ground plane with respect to at least a second of said segments of the perimeter.

2. An antenna arrangement according to claim 1, wherein the substrate overlaps a section of the patch plane along at least one sub-segment of the second of said segments.

3. An antenna arrangement according to claim 1 wherein the grounding member includes a conducting plane which forms a junction with the patch plane along the first of said segments.

4. An antenna arrangement according to claim 1, wherein the grounding member is substantially perpendicular to the ground plane.

5. An antenna arrangement according to claim 1, further comprising a signal member adapted to transfer a radio frequency signal between the patch plane and a radio transceiver.

6. An antenna arrangement according to claim 5, wherein the signal member is electrically insulated from the patch plane.

7. An antenna arrangement according to claim 7 wherein the signal member extends through the ground plane via an electrically insulated feedthrough at a distance from the grounding member.

8. An antenna arrangement according to claim 5 wherein the signal member is electrically connected to a side of the patch plane which faces toward the ground plane.

9. An antenna arrangement according to claim 8 wherein the signal member extends through the ground plane via an electrically insulated feedthrough at a distance from the grounding member (230).

10. An antenna arrangement according to claim 1 wherein the substrate comprises a ceramic material.

11. An antenna arrangement according to claim 1 wherein the substrate comprises a laminated thick-film structure.

12. An antenna arrangement according to claim 11 wherein the laminated thick-film structure includes at least one layer of aluminum oxide.

13. An implantable medical device comprising:
a device casing containing a plurality of electrical components;
an antenna arrangement disposed at an exterior of said casing for communicating radio frequency signals between an exterior of said device casing and at least one of said electrical components, said antenna arrangement comprising:
a patch plane adapted to exchange modulated electromagnetic energy with a surrounding transmission medium, the patch plane having a perimeter formed by a plurality of segments;
a ground plane of an electrically conducting material having an area which is larger than the patch plane, the ground plane being located in relation to the patch plane such that a perpendicular projection of the patch plane onto the ground plane falls entirely within the ground plane;
a dielectric substrate filling a volume between the patch plane and the ground plane;
a grounding member electrically connecting the patch plane to the ground plane, the grounding member being attached to a first of said segments of the perimeter; and
the substrate having high relative permittivity and extending a distance outside the volume between the patch plane and the ground plane with respect to at least a second of said segments of the perimeter.

14. An implantable medical device as claimed in claim 13 wherein said casing has an outer side with a recess therein, and wherein said antenna arrangement is disposed in said recess.

15. An implantable medical device as claimed in claim 13, comprising a non-conductive encapsulation mounted at an exterior of said casing, said antenna arrangement being molded into said non-conducted encapsulation with said ground plane electrically insulated from said casing.

16. An implantable medical device according to claim 13 wherein said casing forms the ground plane.

17. An implantable medical device according to claim 13 wherein the patch plane is tilted with respect to the ground plane such that the patch plane is parallel to the ground plane exclusively in one dimension.

18. An implantable medical device according to claim 17 wherein the grounding member connects the patch plane electrically to the ground plane at a point, and wherein a distance between the patch plane and the ground plane is shortest at said point.

19. An implantable medical device according to claim 18 wherein a portion of the casing forms the ground plane and wherein said portion includes a chamfer having a slope, the chamfer being oriented such that the distance between the patch plane and the ground plane extends gradually along the slope of the chamfer and the distance between the ground plane and the patch plane being largest at an edge thereof farthest from the grounding member.

20. An implantable medical device according to claim 19 wherein the patch plane has a general curved profile with a convex face toward the ground plane.

21. An implantable medical device according to claim 18 wherein the casing has a curved side and wherein the substrate extends over said curved side of the casing such that the substrate forms a tapered section toward the curved side, the curved side being farthest from the grounding member.

22. An implantable medical device according to claim 21 wherein the patch plane has a general curved profile with a convex face toward the ground plane.
